(19) United States
(12) Patent Application Publication
(10) Pub. No.: US 2011/0034870 A1
(43) Pub. Date: Feb. 10, 2011

(54) Drug Mixing and Infusion Device

(75) Inventors: Klaus Glejboel, Glostrup (DK); Morten Revsgaard Frederiksen, Corpenhagen (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 12/373,338

(22) PCT Filed: Jun. 14, 2007
(86) PCT No.: PCT/EP07/55872

§ 371 Date: Dec. 9, 2009

(30) Foreign Application Priority Data
Jul. 13, 2006 (FR) 0652951

(51) Int. Cl. A61M 5/31

(52) U.S. Cl. 604/84

(57) ABSTRACT

A drug mixing device for mixing a lyophilized drug with a liquid, thereby forming a liquid drug and for infusion of the liquid drug. The device comprises a first (1a) and a second (1b) part movable connected to each other and wherein a reservoir (2) is being defined there between for containing a liquid. The first part comprises a vial adapter (3) for a vial-containing a lyophilized drug, and means (13) for establishing a first fluid connection between said reservoir and a vial 44) positioned in the vial adapter and for establishing a second fluid connection (14) between said reservoir and an infusion outlet (15), said means being shiftable between a first position establishing said first fluid connection and interrupting said second fluid connection and a second position interrupting said first fluid connection and establishing said second fluid connection. The second part comprises means (8) for forcing said liquid and/or a solution of liquid and drug between the reservoir, a vial and the infusion outlet.

### FIELD OF THE INVENTION

The present invention relates to a drug mixing and infusion device for storing, reconstituting and infusion of a drug. Further, the present invention relates to a reconstitution device which is easier to operate than similar prior art devices, especially if the person using the device has impaired motor skills or low finger strength.

### BACKGROUND OF THE INVENTION

Mixing and infusion of a freeze dried medication is done using several parts, i.e. a vial containing the dried medication, mixing parts for reconstituting the drug and syringes. This is normally done by means of an ordinary syringe with an ordinary needle. Liquid is sucked into the syringe, the needle is then inserted in a vial containing the lyophilized drug to be reconstituted, and the liquid is forced from the syringe into the vial by means of a movable piston of the syringe. When the lyophilized drug has been properly reconstituted, the movable piston is pulled out, and the reconstituted drug is thereby retrieved to the syringe. The reconstituted drug may subsequently be delivered from the syringe to a person. There is a relatively high risk of contamination of the drug or the syringe during the described procedure due to the exposure of the surfaces and the needle to free air and dirt.

In many prior art reconstitution devices the user is required to deliver a relatively large amount of force in order to operate the device, in particular when performing the part of the operation where a solvent liquid is forced from one reservoir or container into another reservoir or container containing the solid drug. This is a disadvantage, in particular if the user has impaired motor skills or low finger strength.

Various devices for improving the above method of reconstituting lyophilized drug are known. WO 2006/045132 discloses a reconstitution and administering system, having an elongate housing with a forward end located on the discharge side and a rear housing end which lies opposite the forward end. A first chamber containing a lyophilisate is disposed in the area of the forward end inside the housing. The first chamber is tightly sealed at the end facing the forward end by means of a removable closure while being tightly sealed in the direction of the rear housing end with the aid of a plunger. A second chamber is connected to the ambient atmosphere via at least one opening and is embodied in the area of the rear end. The second chamber contains a liquid to be forced into the first chamber to reconstitute the lyophilisate. The liquid is forced into the first chamber by pushing a plunger inside the second chamber whereby a connecting spike generates the fluid connection between the first and second chamber. The reconstituted lyophilisate can be forced out of the first chamber by pushing a plunger inside the first chamber. In the system of WO 2006/045132 it is not possible to move the plunger in both directions which means that the device only can be used to infusion and not aspiration. Furthermore, it is not possible to remove the first chamber after is has been emptied. This is because the design of the system does not allow this. In case a desired dose of medication is larger than what corresponds to the contents of a single vial, it is, however, desirable to be able to remove the vial and replace it with a new filled one (also called “pooling” of vials).

EP 1 329 210 discloses a fluid transfer device for use in the aseptic intermixing of a powder component with a fluid component. The device includes a first adapter that can be connected to a container containing the powder component and a second adapter that can be removably interconnected with the first adapter and can also be connected to a container containing a fluid. When the containers are positioned in the adapters, a fluid connection is established between the containers, thereby allowing fluid from the fluid container to enter the powder container in order to mix the powder and the fluid. However, this device only allows for reconstituting and cannot be used for infusion and aspiration.

U.S. Pat. No. 6,645,171 B1 discloses a device for reconstituting liquid for medical use by bringing together a first liquid medium contained in a first vessel in the form of a cartridge and a second medium, such as a drug in solid form, contained in a second vessel in the form of a vial. The device comprises means for supporting the first and second vessels, and a movable operating member for applying a force to cause the first liquid medium to be delivered at a controlled rate from the first vessel into the second vessel. The first and second vessels may be provided in a pack having liquid transfer means in the form of a needle.

A plunger is provided in the cartridge, and it may be provided with a thread to receive a plunger rod which may be an integral part of the device. The plunger rod may be attached to a lid, and as the lid is screwed into place the pack is compressed. During the compression movement, the housing and the vial move axially towards the cartridge until the needle penetrates the vial, thereby establishing a fluid connection between the two vessels. The liquid is transferred from the first vessel to the second vessel via this fluid connection by means of a compressed spring or a weight. Thereby the force needed to transfer the liquid from the first vessel to the second vessel does not have to be provided by the user, and the device may therefore be operated by a person with impaired motor skills or low finger strength. However, the spring or weight mechanisms disclosed in U.S. Pat. No. 6,645,171 B1 are relatively complicated to use and manufacture.

Further disclosures relevant for the present invention are found in EP 0327519, U.S. Pat. No. 6,478,771, WO 2004052725 and WO 9629113.

### SUMMARY OF THE INVENTION

It is, thus, an object of the invention to provide a device for reconstituting and delivering of drug which is more convenient for the user than prior art devices.

It is, thus, an object of the invention to provide a device for reconstituting a solid drug, the device being easy to use.

It is, thus, an object of the invention to provide one integrated device for both reconstituting and delivering of drug, including both infusion and aspiration.

It is an even further object of the invention to provide a device for reconstituting a solid drug, the device being easily operable by persons with impaired motor skills.

According to the invention the above and other objects are fulfilled by providing a drug mixing device comprising a first and a second part movable connected to each other, a reservoir containing a liquid being defined between the first and second part;

said first part comprising a vial adapter for a vial containing a lyophilized drug, and means for establishing a first fluid connection between said reservoir and a vial positioned in the vial adapter and for establishing a second fluid connection between said reservoir and an infusion outlet, said means being shiftable between a first position establishing said first fluid connection and interrupting said second fluid connection and a second position interrupting said first fluid connection and establishing said second fluid connection, and

said second part comprising means for forcing said liquid and/or a solution of liquid and drug between the reservoir, a vial positioned in the vial adapter and the infusion outlet.

The first and second part may form an at least substantially integral device. In the present context the term ‘integral device’ should be interpreted to mean a device which is manufactured and operated as one device.

The means for establishing a fluid connection can be rotationally operable to provide said fluid connection, and the forcing means can be rotationally operable to force said liquid from the reservoir into the vial.

The second part of the device may comprise a piston element with a plunger provided in the reservoir, which is adapted to force the liquid into the vial upon relative rotational and/or translational movement between the first and second part in a specified direction. The forcing means forces liquid from the reservoir into a vial positioned in the vial adapter via a fluid connection which has been established between the reservoir and the vial. Thereby the lyophilized drug contained in the vial will be reconstituted. The plunger may comprise a rubber seal sealing to inner sidewalls of the first part and thereby seals the reservoir in one end.

The second part with the piston element may be connected to the first part via a threaded engagement, such that the plunger can move in the reservoir by rotationally movement between the first and second part in relation to each other. Alternatively, the first and second may be slidably connected to each other, such that the first and second part moves translatory in relation to each other.

The forcing means may advantageously comprise a plunger or piston which can be rotationally operated, and which is positioned in the reservoir in such a manner that it is movable towards the fluid connections as a result of the rotational operation of the forcing means. Thus, when the forcing means is rotationally operated, the plunger or piston will move in this direction, thereby forcing the liquid through the fluid connection and into the vial.

The means for establishing a fluid connection can be rotationally operable to provide the fluid connection. Thus, when a user wishes to establish a fluid connection, he or she performs a rotational movement of one or more parts of the device, and this rotational movement results in the fluid connection being established. Establishing a fluid connection in a reconstitution device often involves penetrating a septum or a wall of one or more containers or reservoirs. Performing such a penetration by means of a substantially purely linear movement may sometimes require a very high force. However, performing the same penetration by a rotational movement will require a much smaller force. This is a great advantage, in particular if the user has impaired motor skills or low finger strength. Accordingly, it is very advantageous that the means for establishing a fluid connection is rotationally operable.

The forcing means can also be rotationally operable to force the liquid from the reservoir into the vial. Similar to the explanation above, forcing liquid through a fluid connection in order to cause a solid drug to become reconstituted may require a relatively high force if it is performed by means of a substantially purely linear movement. In fact, this operation will often be more demanding than establishing the fluid connection. This may, e.g., be because the fluid connection is relatively narrow and/or because a pressure builds up in the vial as the liquid is transferred, the operation thereby requiring an increasing force during the operation. However, operating the forcing means rotationally reduces the force necessary for forcing the liquid from the reservoir to the vial. Accordingly, this is very advantageous, in particular if the user has impaired motor skills or low finger strength.

Furthermore, simply providing rotationally operable means for establishing a fluid connection and rotationally operable forcing means, results in a device which is mechanically simple, since no complicated constructions are necessary. The device is therefore easy and cost effective to manufacture, and it is easy to use.

The means for establishing a fluid connection may comprise a first threaded connection, said first threaded connection being dialled when the means for establishing a fluid connection is rotationally operated. According to this embodiment, as the thread is dialled a part of the means for establishing a fluid connection will be advanced in a substantially linear movement. However, as described above, the force applied by the user to cause this linear movement is reduced as compared to the situation where a direct linear force is applied, due to the rotational operation. Thus, the means for establishing a fluid connection is, according to this embodiment, simply screwed into position to establish the fluid connection. The threaded portion may advantageously be in the form of a standard luer lock connection. Alternatively or additionally, the forcing means may comprise a second threaded connection, said second threaded connection being dialled when the forcing means is rotationally operated. Similar to what is described above, according to this embodiment, the forcing means is screwed into position to force the liquid from the reservoir into the reservoir. Preferably, the dialling movement may result in a substantially linear movement of a plunger or piston, the plunger or piston thereby forcing the liquid through the fluid connection.

The means for establishing a fluid connection and the forcing means may be simultaneously operable by rotational movement of a single rotatable member. According to this embodiment The means for establishing a fluid connection and the forcing means are either substantially simultaneously or sequentially operable by rotating a single rotational member. This is advantageous because the user in this case only has to concentrate on rotating that single member in order to operate the device, i.e. in order to establish the fluid connection as well as in order to force the liquid from the reservoir into the vial, thereby causing the solid drug to become reconstituted. Thereby the device is very simple and easy to operate.

The device may further comprise releasable locking means for locking the forcing means in a position in which liquid is forced from the reservoir into the vial. Thereby it is prevented that the forcing means moves ‘backwards’ during transfer of the liquid and during the subsequent reconstitution of the solid drug. Accordingly, it is not necessary for the user to apply a constant force to the forcing means during transfer of the liquid, at it is furthermore not necessary for the user to hold the forcing means when the entire amount of liquid has been transferred from the reservoir to the vial, in order to allow the solid drug to reconstitute properly. This is in particular advantageous if the user has impaired motor skills or low finger strength.

The releasable locking means may be or comprise latching means, a snap connection and/or any other kind of means being suitable for locking the forcing means in order to prevent the forcing means from moving ‘backwards’, i.e. for preventing liquid from being transferred from the vial to the reservoir.

Releasing the locking means may cause reconstituted drug to be transferred from the vial into the reservoir via a previously established fluid connection. Thus, according to this embodiment, reconstituted drug will be allowed to be transferred from the vial to the reservoir when the locking means is released. Accordingly, the time of this transfer can be controlled by controlling the time of releasing the locking means. Thus, the liquid can remain in the vial until the solid drug has been properly reconstituted. When a user has established that proper reconstitution has taken place, the locking means is released, and the reconstituted drug is thereby allowed to be transferred back into the reservoir via the fluid connection.

The reconstituted drug may be transferred from the vial into the reservoir due to a substantially linear movement of the forcing means. According to this embodiment it is preferably possible for the forcing means to move linearly relatively to one or more other parts of the device. However, the locking means prevents this movement when it is in a locked position. When the locking means is released, the linear movement of the forcing means is no longer prevented, and the forcing means will move, e.g. as a result of a pressure built up in the vial when the liquid was transferred from the reservoir into the vial.

The locking means may be adapted to be released as a result of a rotational movement of a rotatable member. The rotatable member is preferably also for operating the forcing means, and possibly for operating the means for establishing a fluid connection. In this case the rotatable member is rotated in order to operate the forcing means until the entire amount of liquid has been transferred from the reservoir to the vial. The locking means ensures that the forcing means is locked in this position until the solid drug in the vial has been properly reconstituted. When the user has established that this is the case, the rotatable member is rotated once again. This causes the releasable locking means to be released, and thereby the reconstituted drug is transferred back into the reservoir. In the case that the rotatable member is for releasing the locking means as well as for operating the forcing means, and possibly even the means for establishing a fluid connection, the device will be very simple and easy to use.

Alternatively, the locking means may be adapted to be released in any other suitable manner, e.g. as a result of an at least substantially linear movement. This may, e.g., be obtained by means of a linearly movable member, or by means of a push button.

The connection between the first and second part allows for movement of the plunger both in the direction forcing the liquid into the vial and also in the opposite direction sucking a solution of liquid and drug from the vial and back into the reservoir. The device can thereby also be used to aspiration via the infusion outlet when the second fluid connection is established by moving the plunger in its sucking direction sucking blood into an infusion needle positioned in the vein and connected to the infusion outlet.

The liquid contained in the reservoir is preferably a solvent liquid being suitable for reconstitution of a lyophilized drug contained in the vial. The amount and kind of liquid in the reservoir is preferably chosen in such a manner that it matches a specific lyophilized drug. Thereby it is ensured, that when a vial containing that specific lyophilized drug is positioned in the vial adapter, the lyophilized drug is reconstituted correctly and in a suitable manner.

The vial adapter is a part of the device which is shaped in such a manner that a vial may be positioned there in a manner which substantially fixes the vial relatively to the device, and in such a manner that access may be gained to the interior of the vial. Thus, the vial adapter may comprise means for gripping a vial, e.g. a neck or shoulder part of a vial.

The vial containing the lyophilised drug may be a separate part to be positioned in the device, or it may be contained in the device and positioned in or forming part of the vial adapter. If the vial is removable, it is possible to constitute a dose of medication using reconstituted drug from two or more vials, i.e. it is possible to ‘pool’ the contents of two or more vials. This may be done by retrieving the reconstituted drug to the reservoir, replacing the vial with a new filled one and subsequently reconstitute the content of new vial. Thereby the total dose which is eventually contained in the reservoir will correspond to the combined dose of all the vials used. The total dose may then be delivered directly from the reservoir. Thus, by making it possible to remove the vial from the device, pooling of the contents of two or more vials is made easier.

Preferably, the means for establishing a fluid connection between the vial and reservoir comprises a valve system to be positioned in at least said first position providing fluid connection between the vial and reservoir and said second position providing fluid connection between the reservoir and the infusion outlet.

Preferably, the means for establishing a fluid connection between the vial and the reservoir further comprises a first spike positioned at or near the vial adapter and being adapted to penetrate a septum of a vial positioned in the vial adapter. Thus, the first spike is preferably a hollow spike giving access to the interior of the vial when pressing the vial towards the spike for penetrating the septum, and the first spike may in combination with the valve system provide the fluid connection between the vial and reservoir.

However, the means for establishing a fluid connection between the vial and reservoir may further comprise a second spike adapted to penetrate the reservoir. The first and second spike may for example form part of a double pointed hollow needle. In this case the first spike will penetrate the septum of the vial and the second spike will penetrate a septum/sealing of the reservoir, the double pointed hollow needle thereby establishing the fluid connection between the vial and reservoir. The double pointed needle may in combination with the valve system provide the fluid connection between the vial and reservoir, meaning that the valve can be positioned in the first position after the first and/or second spike has penetrated the septum of the vial and reservoir, respectively. When the valve is positioned in the first position, the liquid can be forced into the vial by means of the plunger and the drug is reconstituted. Then, the solution can be sucked back into the reservoir by means of the plunger, as described above, and the valve system can then be positioned in the second position allowing the solution to be injected directly from the reservoir via the infusion outlet and an infusion needle to the user. Before injecting the drug, the user may aspirate by moving the first and second part in relation to each other so that the plunger via the second fluid connection sucks blood into the infusion needle.

The valve system may comprise a slide valve being slidable between said two positions whereby the valve closes the fluid connection between the vial and the reservoir when being moved from its first position to its second position, and vice versa. The valve system may comprise any valve type, such as a turning valve.

The valve system may comprise a syringe having two internal channels, a first channel for the first fluid connection between the vial and reservoir, and a second channel for the second fluid connection. The second channel extends from an inlet adjacent to the reservoir to an outlet forming the infusion outlet, said outlet being connectable to an infusion needle, such as a butterfly needle.

A penetrable seal may be provided over said first spike and/or second spike.

The present invention also relates to a drug mixing and infusion kit comprising a drug mixing device as described above and an infusion needle to be connected to the infusion outlet, said needle preferably being a butterfly needle.

Thus, the invention provides a device which may be delivered as an ‘all-in-one’ and/or a ‘ready-to-use’ package including all parts needed for reconstituting and infusing the drug and which is easy to operate. It is only necessary to place an appropriate vial in the vial adapter, place a needle at the infusion outlet, operate the means for establishing the fluid connections and the forcing means to reconstitute the drug and infuse it.

### FEATURES OF THE INVENTION

1. A drug mixing device comprising a first and a second part movable connected to each other, a reservoir containing a liquid being defined between the first and second part;
said first part comprising a vial adapter for a vial containing a lyophilized drug, and a valve system for establishing a first fluid connection between said reservoir and a vial positioned in the vial adapter and for establishing a second fluid connection between said reservoir and an infusion outlet, said valve system being shiftable between a first position establishing said first fluid connection and interrupting said second fluid connection and a second position interrupting said first fluid connection and establishing said second fluid connection,
said second part comprising means for forcing and/or sucking said liquid and/or a solution of liquid and drug between the reservoir, a vial positioned in the vial adapter and the infusion outlet.

2. A drug mixing device according to feature 1, wherein the first and second part form an at least substantially integral device.

3. A drug mixing device according to feature 1 or 2, further comprising a vial containing a lyophilized drug, the vial being positioned in or forming part of the vial adapter.

4. A drug mixing device according to any of the preceding features, wherein the valve system comprises a slide valve being slidable between said two positions or a turning valve or any other valve type.

5. A drug mixing device according to any of the preceding features, wherein the means for establishing a fluid connection between the vial and the reservoir further comprises a first spike positioned at or near the vial adapter and being adapted to penetrate a septum of a vial positioned in the vial adapter.

6. A drug mixing device according to any of the preceding features wherein the means for establishing a fluid connection between the vial and reservoir further comprises a second spike adapted to penetrate the reservoir.

7. A drug mixing device according to any of the preceding features, wherein the forcing means comprises a plunger provided in the reservoir and being adapted to force the liquid into the vial upon relative rotational and/or translatory movement between the first and second part in a specified direction.

8. A drug mixing device according to feature 7, wherein further the means for establishing a fluid connection is rotationally operable to provide said fluid connection.

9. A drug mixing device according to feature 8, wherein the means for establishing a fluid connection comprises a first threaded connection, said first threaded connection being dialled when the means for establishing a fluid connection is rotationally operated.

10. A drug mixing device according to any of the features 7-9, wherein the forcing means comprises a second threaded connection, said second threaded connection being dialled when the forcing means is rotationally operated.

11. A drug mixing device according to any of the features 8-10, wherein the means for establishing a fluid connection and the forcing means are simultaneously operable by rotational movement of a single rotatable member.

12. A drug mixing device according to any of the preceding features, further comprising releasable locking means for locking the forcing means in a position in which liquid is forced from the reservoir into the vial.

13. A drug mixing device according to feature 12, wherein releasing the locking means causes reconstituted drug to be transferred from the vial into the reservoir via a previously established fluid connection.

14. A drug mixing device according to feature 13, wherein the reconstituted drug is transferred from the vial into the reservoir due to a substantially linear movement of the forcing means.

15. A drug mixing device according to any of the features 12-14, wherein the locking means is adapted to be released as a result of a rotational movement of a rotatable member.

16. A drug mixing device according to any of features 5-15, further comprising a penetrable seal provided over said first spike and/or second spike.

17. A drug mixing and infusion kit comprising a drug mixing device according to any of features 1-16 and an infusion needle to be connected to the infusion outlet.

18. A drug mixing and infusion kit according to feature 17, wherein the forcing means is adapted to suck, via said infusion outlet, blood into the needle (aspiration) upon relative rotational and/or translatory movement between the first and second part in a specified direction.

### BRIEF DESCRIPTION OF THE DRAWINGS

The invention will now be described in further details with reference to the accompanying drawings in which

FIG. 1a-b shows a drug mixing and infusion device according to an embodiment of the invention,

FIG. 2 is a cross-sectional view of a drug mixing and infusion device according to another embodiment of the invention, and

FIG. 3 is a cross-sectional view of a drug mixing and infusion device according to another embodiment of the invention

FIG. 4 is illustrating a cross sectional view of a device according to an embodiment of the invention.

### DETAILED DESCRIPTION OF THE DRAWINGS

FIG. 1a-b shows a drug mixing and infusion device according to an embodiment of the invention. The device comprises two main parts; a first part 1a comprising a vial adapter 3 and a valve system 12 for establishing fluid connections, and a second part 1b comprising a piston element 8 with a plunger 9. A reservoir 2 containing a liquid is defined between the first part 1a and the plunger 9 of the piston element 8. At the vial adapter 3 holding a vial 4 there is positioned a first hollow spike 5 adapted to penetrate a seal 6 on the spike itself and a septum 7 of the vial 4.

The piston element 8 having a plunger 9 is movable inside the reservoir 2 when rotating the second part 1b in relation to the first part 1a of the device via the threaded engagement 10 in the direction 11, either clock-wise or counter clock-wise depending on whether the plunger shall force liquid out of the reservoir or suck it back into the reservoir.

The valve system 12, in the form of a syringe-like member, is provided between the vial 4 and the reservoir 2. The valve system 12 comprises a slide valve being shiftable between a first position establishing a first fluid connection 13 between the reservoir 2 and vial 4 and interrupting a second fluid connection 14 between the reservoir 2 and an infusion outlet 15 and a second position interrupting said first fluid connection 13 and establishing said second fluid connection 14.

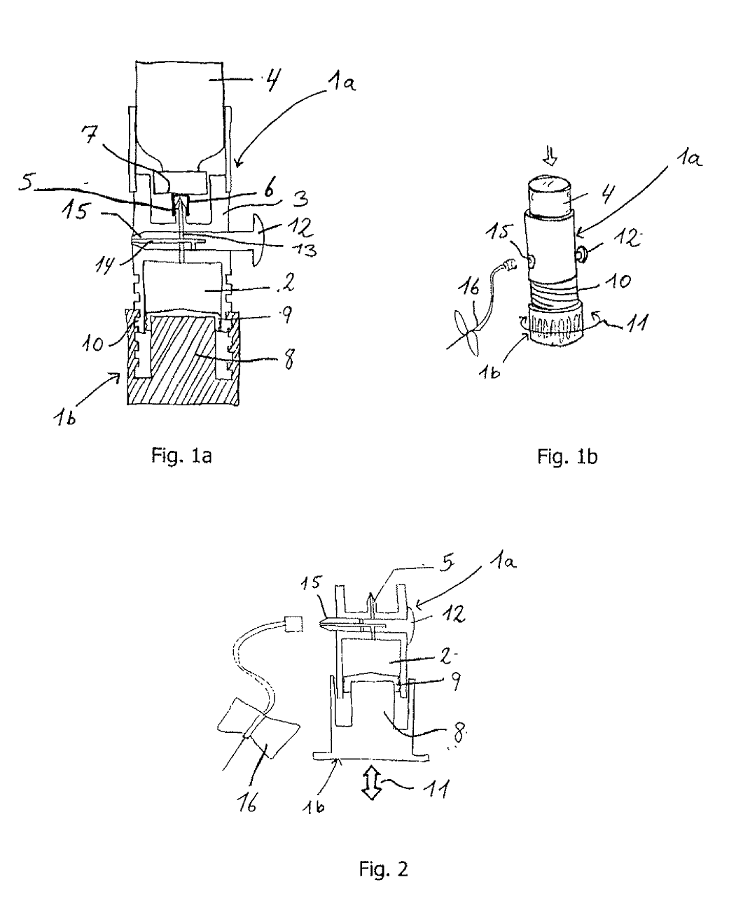

By moving the vial 4 towards the spike 5, the spike 5 penetrates the seal and septum 6, 7, and a first fluid connection 12 is provided between the vial 4 and the reservoir 2 through the hollow spike 5 and valve 12, when the valve 12 is in said first position as shown in FIG. 1a. Then the piston element 8 is rotated in relation to the other part of the device resulting in a movement of the plunger 9 towards the vial, which then forces the liquid into the vial 4. The lyophilized drug in the vial is reconstituted, and it can be sucked back into the reservoir by rotating the piston element 8 in the opposite direction. When the reconstituted drug is sucked into the reservoir 2, the valve 12 is slit to its second position (as shown in FIG. 2) providing the second fluid connection 14 between the reservoir 2 and the infusion outlet 15. A butterfly needle 16 is connected to the outlet 15, and the user can infuse the drug by rotating the piston element 8 counter-clockwise and thereby forcing the reconstituted drug out through the infusion outlet 15. Prior to infuse, the user may aspirate through the butterfly needle by rotating the piston element 8 clockwise to provide a suction process in the needle.

FIG. 1b is a perspective view of the device of FIG. 1a.

FIG. 2 is a cross-sectional view of a drug mixing and infusion device according to another embodiment of the invention. The embodiment of FIG. 2 is similar to the embodiment of FIGS. 1a-b except for the engagement and thereby movement between the first and second part of the device. Instead of having a threaded engagement, the parts slide by translation in relation to each other, so that the plunger 9 forces the liquid out of the reservoir by pressing the piston element 8 in the direction 11, and vice versa.

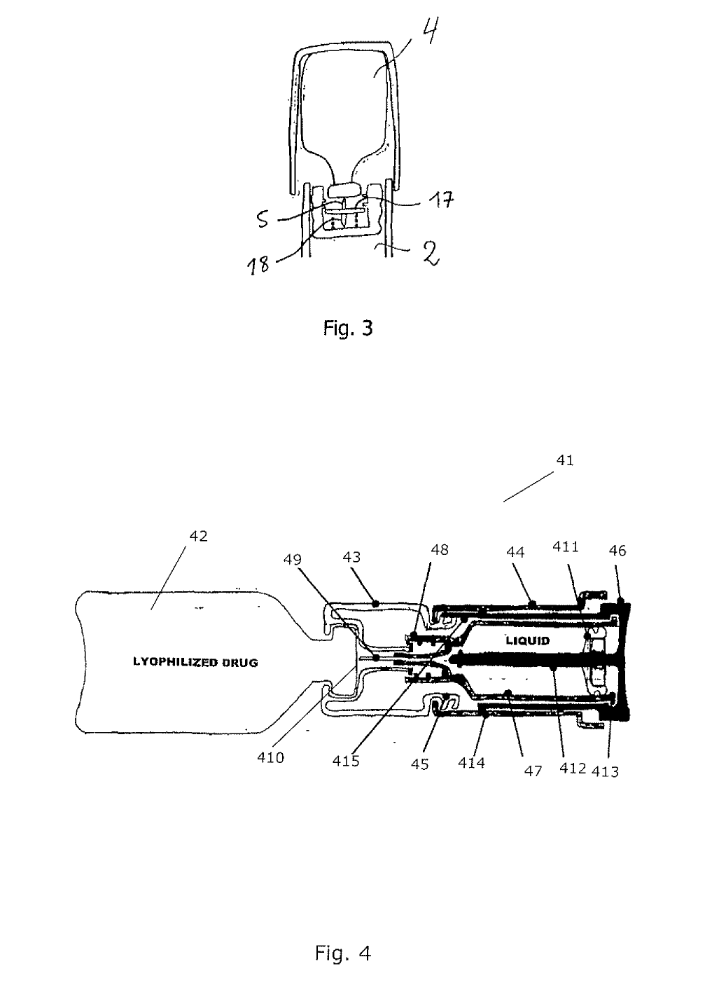

FIG. 3 is a cross-sectional view of a drug mixing and infusion device according to another embodiment of the invention. The embodiment is similar to the embodiments of FIGS. 1a-b and 2, except for the means for establishing fluid connections between the vial 4 and reservoir 2. The vial adapter comprises a double needle 17 consisting of a first spike 5 and a second spike 18. The first spike 5 will penetrate the septum 7 of the vial and the second spike 18 will penetrate a septum/sealing of the reservoir 2, the double pointed hollow needle thereby establishing the fluid connection between the vial and reservoir. The valve system is not shown in FIG. 3, but it will in combination with the double pointed needle 17 provide the fluid connection between the vial and reservoir, meaning that the valve can be positioned in the first position after the first and/or second spike has penetrated the septum of the vial and reservoir, respectively.

FIG. 4 is a cross sectional view of a device 41 according to an embodiment of the invention. The device 41 comprises a vial in the form of a vial 42 positioned in a receiving means 43. The receiving means 43 is detachably assembled to a syringe part 4 of the device 41 via a threaded portion 45. The syringe part 44 and the vial 42 may rotate relatively to each other in a direction opposite the direction of the threaded portion 45. This will be explained in further details below.

Inside the syringe part 44 there is a plunger rod 46, and inside the plunger rod 46 there is a reservoir 47 containing a liquid which is suitable for reconstituting the solid drug in the vial 42. The reservoir 47 is provided with a luer lock connection 48. In front of the luer lock connection 48 there is positioned a hollow needle 49 which is adapted to penetrate a septum 410 of the vial 42, thereby establishing a fluid connection between the reservoir 47 and the vial 42.

In the reservoir 47 there is positioned a plunger 411 which is movable along a threaded plunger rod 412. As the plunger 411 moves along the threaded plunger rod 412 it pushes liquid through the hollow needle 49 and into the vial 42, thereby causing the solid drug in the vial 42 to become reconstituted.

The reservoir 47 is provided with a snap connection 413 which locks the plunger rod 46 axially to the reservoir 47. Accordingly, the plunger rod 46 is not allowed to move in a direction away from the vial 42, i.e. to the right in the Figure, as long as the snap connection 413 is locked. Thereby the plunger 411 is also prevented from moving in that direction and liquid can therefore not return from the vial 42 to the reservoir 47. However, when the snap connection 413 is released the plunger rod 46 is free to move in the direction defined above along a linear slot 414 formed in the syringe part 44. This will be explained in further details below.

The device 41 illustrated in FIG. 4 is preferably operated in the following manner. The device 41 is delivered with the parts of the device 41 positioned relatively to each other as shown in FIG. 4. The syringe part 44 is rotated in a clockwise direction relatively to the receiving means 43. Due to the linear slot 414 this will also cause the plunger rod 46 to rotate in a clockwise direction. Furthermore, the reservoir 47 will be rotated along in the following manner. Due to a protruding part on the threaded plunger rod 412 (not visible in the Figure), the plunger 411 is rotated along with the threaded plunger rod 412. This rotational movement is transferred to the reservoir 47 due to friction between the plunger 411 and the reservoir 47. In an alternative embodiment, the rotational movement may be transferred to the reservoir 47 by means of a linear track between the plunger rod 6 and the reservoir 47. Thus, a threaded portion positioned inside the luer lock connection 48 is dialled, thereby causing the luer lock connection 48 to move in a direction towards the vial 42. The plunger rod 46, the threaded plunger rod 412, the reservoir 47 and the plunger 411 are moved along with the luer lock connection 48. Since the hollow needle 49 is positioned in front of the luer lock connection 48 it will also be pushed towards the vial 42, thereby penetrating the septum 410 and establishing a fluid connection between the vial 42 and the reservoir 47. Continued clockwise rotation of the syringe part 44 causes the plunger rod 46, and thereby the threaded plunger rod 412 to rotate, thereby moving the plunger 411 in a direction towards the vial 42, i.e. to the left in the Figure. Accordingly, liquid is pushed by the plunger 411 from the reservoir 47 into the vial 42 via the hollow needle 49. It should be noted that the luer lock connection 48 in conjunction with the hollow needle 49 and the septum 410 functions as a valve, ensuring that the reservoir 47 and the vial 42 are properly connected before liquid is being pushed out of the reservoir 47.

When the plunger 411 reaches a position where the entire amount of liquid has been transferred from the reservoir 47 to the vial 42 the torque increases, and the user will feel an increased resistance. Thereby the user will know that the entire amount of liquid has been transferred to the vial 42, and that reconstitution of the solid drug is now taking place. The engagement between the plunger 411 and the threaded plunger rod 412, as well as the snap connection 413 ensures that the plunger 411 remains in this position, i.e. the liquid is prevented from returning to the reservoir 47. Accordingly, the user does not have to apply a force in order to ensure this while the solid drug is being reconstituted, and it is therefore easy for the user to monitor the reconstitution.

When the user has established that proper reconstitution has taken place, a higher clockwise torque is applied to the syringe part 44. Since the plunger 6 is not able to move any further in the direction towards the vial 42, i.e. to the left in the Figure, this will cause the threaded plunger rod 412, and thereby the plunger rod 46, to move in the opposite direction, i.e. to the right in the Figure. This will cause the snap connection 413 to be released from its locked position, thereby releasing the plunger rod 6. Accordingly, the plunger rod 46, the threaded plunger rod 412 and the plunger 411 will move in a direction away from the vial 42, i.e. to the right in the Figure, along the linear slot 414, due the pressure built up in the vial 42 during the transfer of the liquid.

So far counter-clockwise rotation of the syringe part 44 relatively to the receiving means 43 has been prevented by locking pin 415. However, as plunger rod 6 moves in a direction away from the vial 42 as described above, locking pin 415 also moves in this direction, and it is thereby possible to perform counter-clockwise rotation of the syringe part 44 relatively to the receiving means 43.

When the reconstituted drug has been fully transferred to the reservoir 47, the receiving means 43 and the syringe part 44 are detached at the threaded portion 45 by performing a counter-clockwise rotation. Thereby the luer lock connection 48 is exposed. The hollow needle 49 will remain in the septum 410, and will thereby be positioned and protected inside the receiving means 43. Thereby the risk of injuries due to needle sticks is reduced.

The luer lock connection 48 can now be attached to suitable delivering means, such as a syringe needle, a butterfly needle, relevant tubing, etc. Once this is done, the syringe part 44 is ready for delivering the reconstituted drug via the luer lock connection 48, simply by pressing the plunger rod 46.

1. A drug mixing device comprising:
a first part and a second part movable connected to each other,
a reservoir containing a liquid being defined between the first and second part;
said first part comprising a vial adapter adapted to substantially fix a vial containing a lyophilized drug relative to the device, and a valve system for establishing a first fluid connection between said reservoir and a vial positioned in the vial adapter and for establishing a second fluid connection between said reservoir and an infusion outlet, said valve system being shiftable between a first position establishing said first fluid connection and interrupting said second fluid connection, and a second position interrupting said first fluid connection and establishing said second fluid connection,
said second part comprising means for forcing and/or sucking said liquid and/or a solution of liquid and drug between the reservoir, a vial positioned in the vial adapter and the infusion outlet.

2. A drug mixing device according to claim 1, wherein the first part and second part form an at least substantially integral device.

3. A drug mixing device according to claim 1, further comprising a vial containing a lyophilized drug, the vial being positioned in or forming part of the vial adapter.

4. A drug mixing device according to claim 1, wherein the valve system comprises a slide valve being slidable between said two positions or a turning valve.

5. A drug mixing device according to claim 1, wherein the means for establishing a fluid connection between the vial and the reservoir further comprises a first spike positioned at or near the vial adapter and being adapted to penetrate a septum of a vial positioned in the vial adapter.

6. A drug mixing device according to claim 1, wherein the means for establishing a fluid connection between the vial and reservoir further comprises a second spike adapted to penetrate the reservoir.

7. A drug mixing device according to claim 1, wherein the forcing means comprises a plunger provided in the reservoir and being adapted to force the liquid into the vial upon relative rotational and/or translatory movement between the first and second part in a specified direction.

8. A drug mixing device according to claim 7, wherein further the means for establishing a fluid connection is rotationally operable to provide said fluid connection.

9. A drug mixing device according to claim 8, wherein the means for establishing a fluid connection comprises a first threaded connection, said first threaded connection being dialed when the means for establishing a fluid connection is rotationally operated.

10. A drug mixing device according to claim 7, wherein the forcing means comprises a second threaded connection, said second threaded connection being dialed when the forcing means is rotationally operated.

11. A drug mixing device according to claim 8, wherein the means for establishing a fluid connection and the forcing means are simultaneously operable by rotational movement of a single rotatable member.

12. A drug mixing device according to claim 1, further comprising releasable locking means for locking the forcing means in a position in which liquid is adapted to be forced from the reservoir into the vial.

13. A drug mixing device according to claim 12, wherein releasing the locking means causes reconstituted drug to be transferred from the vial into the reservoir via a previously established fluid connection when a vial containing a lyophilized drug is present.

14. A drug mixing device according to claim 13, wherein the reconstituted drug is transferred from the vial into the reservoir due to a substantially linear movement of the forcing means.

15. A drug mixing device according to claim 12, wherein the locking means is adapted to be released as a result of a rotational movement of a rotatable member.

16. A drug mixing device according to claim 6, further comprising a penetrable seal provided over said first spike and/or said second spike.

17. A drug mixing and infusion kit comprising:
a drug mixing device according to claim 1 and an infusion needle adapted to be connected to the infusion outlet.

18. A drug mixing and infusion kit according to claim 17, wherein the forcing means is adapted to suck, via said infusion outlet, blood into the needle (aspiration) upon relative rotational and/or translatory movement between the first and second part in a specified direction.
